(19) United States
(12) Patent Application Publication
(10) Pub. No.: US 2016/0053226 A1
(43) Pub. Date: Feb. 25, 2016

(54) Method and Medium for Amplifying Neural Precursor Cells

(71) Applicants: INSERM (INSTITUT NATIONAL DE LA SANTÉ ET DE LA RECHERCHE MÉDICALE, Paris (FR); UNIVERSITY D'EVRY VAL D'ESSONE, Evry (FR); ASSOCIATION FRANCAISE CONTRE LES MYOPATHIES, Paris (FR)

(72) Inventors: Marc Peschanski, Evry Cedex (FR); Alexandra Benchoua, Evry (FR)

(21) Appl. No.: 14/784,627

(22) PCT Filed: Apr. 15, 2014
(86) PCT No.: PCT/EP2014/057658

§ 371 Date: Oct. 15, 2015

(30) Foreign Application Priority Data
Apr. 15, 2013 (EP) 13305489.0

(51) Int. Cl. C12N 5/0797; A61K 35/30; G01N 33/50

(52) U.S. Cl. CPC C12N 5/0623; G01N 33/5058; A61K 35/30

(57) ABSTRACT

The present invention relates to a method for amplifying a population of neural precursors comprising the step of culturing neural precursors in the presence of a PKA inhibitor.

### FIELD OF THE INVENTION

The invention relates to a method and a medium for the amplification of neural precursor cells, in particular neural precursor cells derived from pluripotent stem cells.

### BACKGROUND OF THE INVENTION

Pluripotent stem cells (PSC), present two advantages due to their intrinsic properties: firstly they can provide a quasi-unlimited pool of cells due to their self-renewal capacity, and secondly, they are capable of differentiating in vitro into any cell lineage, including all the neural lineage cells types.

Production of neuronal and glial cells from PSC promises to be an invaluable tool for establishing in vitro cellular models in order to study neurological or psychiatric diseases, as well as for the development of cell therapy-based strategies for certain neurological conditions.

The phenotypic transition from stem cells to neural precursors represents a limiting and crucial step of the neural, and later neuronal and glial, differentiation process. Recently, techniques for obtaining neural precursors from PSC have been described. For instance, WO2010/063848 describes a method for producing a population of neural precursors wherein PSC are cultured in the presence of an inhibitor of the BMP signalling pathway, such as Noggin, and of an inhibitor of the TGF/activin/Nodal signalling pathway, such as SB431542.

These methods provide limited amounts of neural precursors, which then need to be amplified, i.e. cultured under conditions which allow the symmetrical division of the neural presursors without losing their ability to differentiate into any kind of neural lineage cell type.

Conti et al., (2005) have described a method for amplifying a population of neural precursors, wherein said neural precursors are cultured in the presence of fibroblast growth factor 2 (FGF-2) and epidermal growth factor (EGF). Other authors (Zhang et al., 2011; Chen et al., 2013), have found that BDNF could also be used, in combination with FGF2 and EGF, in order to promote cell survival.

However, these methods present several drawbacks, due to the presence of protein growth factors: high costs, a batch-dependent efficacy, and hurdles for adapting protocols in order to meet the requirements of Good Manufacturing Practice (GMP) conditions and/or to provide products useful for cell therapy.

Thus, there is still a need in the art for alternative methods for amplifying neural precursors, and in particular human neural precursors.

### SUMMARY OF THE INVENTION

The present invention relates to a method for amplifying a population of neural precursors comprising the step of culturing neural precursors in the presence of a PKA inhibitor.

The present invention also relates to the use of a PKA inhibitor for amplifying a population of neural precursors.

Also provided is a kit for the culture of neural precursors, comprising a culture medium and a PKA inhibitor.

Also provided is a population of neural precursors obtainable by the method of the invention.

In another aspect, the invention also relates to a method for obtaining a population of neurons wherein said method comprises the steps of:

a. obtaining a population of neural precursors

b. amplifying said population of neural precursors in the presence of a PKA inhibitor

c. differentiating said population of neural stem cells into neurons.

In another aspect, the invention also provides a method for screening compounds having a neuroprotective and/or neurotoxic effect wherein said method comprises the steps of:

a) culturing a population of neural precursors or a population of neurons as described above in the presence of a test compound;

b) comparing the survival of the cells cultured in step a) to that of a population of neural precursors or a population of neurons as defined above cultured in the absence of said test compound.

### DETAILED DESCRIPTION OF THE INVENTION

The present invention relates to a method for amplifying a population of neural precursors comprising the step of culturing neural precursors in the presence of a PKA inhibitor.

The term “PKA inhibitor” as used herein refers to any compound, natural or synthetic, which inhibits the serine-threonine kinase activity of PKA

The skilled person in the art knows how to assess whether a given compound is a PKA inhibitor. Typically, a compound can be tested for its ability to inhibit cAMP-dependent phosphate transfer on a LRRASLG peptide substrate in presence of recombinant Protein Kinase A. This can be done for instance with the SignaTECT® cAMP-Dependent Protein Kinase (PKA) Assay System (available from Promega under the reference V7480).

In one embodiment of the invention, the PKA inhibitor is a selective PKA inhibitor.

Suitable selective inhibitors of PKA according to the invention can be 1) structural analogs of cAMP, the Rp-cAMPs family, that are competitive inhibitors of the cAMP-binding site, 2) endogeneous inhibitors of the activity of PKA: the protein kinase inhibitor peptide (Murray, 2008) and 3) silencing RNA targeted to at least the two isoforms of the catalytic subunit of PKA (Murray, 2008; Dumaz et al, 2003; Rudolph et al, 2007; Monagham et al, 2008). These compounds include, but are not limited to Rp-adenosine-3′,5′-cyclic monophosphorothioate (Wang et al, 1991); Rp-8-Bromoadenosine 3′,5′-cyclic Monophosphorothioate (Gjertsen et al, 1995); Rp-8-bromo-2′-O-monobutyryladenosine-3′,5′-cyclic monophosphorothioate (Ruiz-Velasco et al, 1998); Rp-8-chloroadenosine-3 ‘,5’-cyclic monophosphorothioate (Yokozaki et al, 1992); Rp-8-(4-chlorophenylthio)adenosine-3′,5′-cyclic monophosphorothioate (Weisskupf et al, 1994); Rp-8-hexylaminoadenosine-3′,5′-cyclic monophosphorothioate (Gjertsen et al, 1995); Rp-8-hydroxyadenosine-3 ‘,5’-cyclic monophosphorothioate (Gjertsen et al, 1995); Rp-8-PIP-cAMPs (Ogreid et al, 1994); PKA inhibitor fragment 14-22 myristoylated (Zhang et al, 2004); PKA inhibitor fragment 6-22 amide (Glass et al, 1989); PKA inhibitor fragment 5-22 amide (Cheng et al, 1986); cAMP-dependent protein kinase inhibitor fragment 5-24, (Cheng et al, 1986).

According to another embodiment of the invention the PKA kinase inhibitor is a non-selective kinase inhibitor, i.e. a compound which inhibits at least one other kinase in addition to PKA. Suitable non-selective PKA kinase inhibitors include, but are not limited to H89 (Murray, 2008); KT5720 (Murray, 2008); chelerythrine (Freemerman et al, 1996); H7 (Qiu et al, 2010); H8 (Hidaka et al, 1984); protein kinase inhibitor from porcine heart; H9, HA1077 and HA1004.

Typically, the non-selective kinase inhibitor can be H89, which can be purchased from Sigma-Aldrich under reference B1427.

Typically, the non-selective kinase inhibitor can be HA1004, which can be purchased from under reference

In one embodiment of the invention, the PKA inhibitor is selected from the group consisting of H7, H8, H9, H89, HA1077 and HA 1004.

In a preferred embodiment, said PKA inhibitor is HA 1004.

HA 1004, also known as, N-(2′-Guanidinoethyl)-5-isoquinolinesulfonamide.HCl, is commercially available. For instance, HA 1004 can be purchased from Enzo Life Sciences under the reference BML-EI184-0010.

It falls within the ability of the skilled person to determine the optimal concentration of the PKA inhibitor in the final culture medium.

Typically, the PKA inhibitor of the invention can be used at a concentration comprised between 1 and 100 μM, preferably between 5 and 80, even more preferably between 20 and 40 μM.

In one embodiment, the PKA inhibitor is HA 1004 and is used at a concentration of about 20 to about 40 μM in the culture medium.

The term “culture medium” as used herein refers to a liquid medium suitable for the in vitro culture of mammalian cells. Typically, the culture medium of the invention contains:
a source of carbon as energy substrate, such as glucose, galactose or sodium pyruvate;
essential amino-acids;
vitamins, such as biotin, folic acid, B12 . . . ;
at least a purine and a pyrimidine as nucleic acid precursors;
inorganic salts;
a molecule known to limit natural ageing, such as selenium;
an antioxidant, such as glutathione reduced (GSH), Ascorbic acid, or enzymes involved in Reactive Oxygen Species detoxification, such as catalase or superoxide dismutase;
a phospholipid precursor, such as choline, inositol or cholesterol derivative such as corticosterone;
an unique fatty acid, such as linoleic acid, linolenic acid and/or lipoic acids;
a carrier protein, such as Albumin and/or Heparin;
optionally, other proteins or peptides, such as insulin and/or transferrin and/or an agonist of the IGF-1 receptor.

The culture medium may also contain pH buffers in order to maintain the pH of the medium at a value suitable for cell growth.

The culture medium of the invention may be based on a commercially available medium such as DMEM/F12 from Invitrogen or a mixture of DMEM/F12 and Neurobasal in a 1:1 ratio (also from Invitrogen).

The culture medium of the invention may also comprise various supplements such as B-27 supplement (Invitrogen) and N2 supplement (also from Invitrogen).

The B27 supplement contains, amongst other constituents, SOD, catalase and other anti-oxidants (GSH), and unique fatty acids, such as linoleic acid, linolenic acid, lipoic acids.

The N2 supplement can be replaced with the following cocktail: transferrin (10 g/L), insulin (500 mg/L), progesterone (0.63 mg/L), putrescine (1611 mg/L) and selenite (0.52 mg/L).

The term “N2B27” refers to the medium described in Ying et al., 2003, in Lowell et al., 2006 and in Liu Y et al., 2006. N2B27 comprises DMEM/F12 and Neurobasal media in a 1/1 ratio, N2 supplement (1/100), B27 supplement (1/50) and beta-mercaptoethanol (1/1000). It is available, for example, under reference SCS-SF-NB-02 from Stem Cell Sciences UK Ltd.

Typically, the culture medium of the invention is free of serum and free of serum extract.

In a preferred embodiment, the culture medium of the invention is free of animal-derived substances. In a preferred embodiment, the culture medium of the invention consists essentially of synthetic compounds, compounds of human origin and water. Advantageously, said culture medium can be used for culturing cells according to good manufacturing practices (under “GMP” conditions).

The invention also relates to the use of a PKA inhibitor for amplifying neural precursors.

The invention also relates to a kit for cell culture comprising a medium base (such as the N2B27 medium defined above), and a PKA inhibitor as described above.

The term “neural precursors or neural stem cells” as used herein refers to cells which are engaged in the neural lineage and which can give rise to any cell of the neural lineage including neurons and glial cells. Typically, neural precursors express the following markers: SOX1, SOX2, PAX6, Nestin, N-CAM (CD56), see Tabar et al., 2005; Sun et al., 2008.

Neural precursors can be obtained from a wide variety of sources.

They can be derived directly from embryos, from adult tissue, from foetal tissue, from embryonic stem (ES) cells (either wild-type ES cells or ES cells carrying a mutation), ES cell lines or induced pluripotent stem cells (iPS cells).

Neural precursors can be obtained from any mammalian species, including, but not limited to humans, primates, rodents (rat, mouse etc.), dogs, cats or felines.

Typically, neural precursors can be obtained by differentiating pluripotent cells into neural precursors.

The term “pluripotent cells” as used herein refers to undifferentiated cells which can give rise to a variety of different cell lineages. Typically, pluripotent cells may express the following markers oct4, SOX2, Nanog, SSEA 3 and 4, TRA 1/81, see International Stem Cell Initiative recommendations, 2007.

In one embodiment, the pluripotent cells are human pluripotent cells.

In another embodiment, the pluripotent cells are non-human mammalian pluripotent cells.

In one embodiment, the pluripotent cells are stem cells.

Typically, said stem cells are embryonic stem cells.

In a preferred embodiment, the pluripotent cells are human embryonic stem cells (hES cells). Typically, hES cell lines such as the one described in the following table may be employed for the method of the invention:

| | | passage | country of | |
|---|---|---|---|---|
| line | karyotype | available | origin | origin |
| SA01 | 46XY | 25 | Sweden | Cellartis AB |
| VUB01 | 46XY | 73 | Belgium | AZ-VUB Bruxel |
| HUES 24, | 46XY | 26 | USA | Harvard |
| H1 | 46XY, | 26 | USA | Wicell research |
| | 20q11.21 | | | Institute |
| H9 | 46XX | 27 | USA | Wicell research |
| | | | | Institute |
| WT3 | 46XY | 35 | UK | UKSCB |

In one embodiment, the pluripotent cells are non-human embryonic stem cells, such a mouse stem cells.

In one embodiment, the pluripotent cells are induced pluripotent stem cells (iPS). Induced pluripotent stem cells (iPS cells) are a type of pluripotent stem cells artificially derived from a non-pluripotent, typically an adult somatic cell, by inducing a “forced” expression of certain genes. iPS cells were first produced in 2006 from mouse cells (Takahashi et al Cell 2006 126:663-76) and in 2007 from human cells (Takahashi et al. Cell 2007 131-861-72, Yu et al. Science 2007 318:1917).

In one embodiment, the pluripotent cells contain a genetic mutation responsible for a neurodegenerative genetic disease. Advantageously, in this embodiment, the population of neural precursors obtained from said pluripotent cells also contains said mutation and can therefore provide a good cellular model of the disease.

Typically, cells lines baring triplet mutations causing the following neurodegenerative diseases can be employed:

| Triplet mutation | Disease | Line | Reference |
|---|---|---|---|
| Huntingtin-CAG | Huntington's | VUB05 | K. sermon, AZ-VUB |
| | disease | | Bruxel BELGIUM |
| Ataxin7-CAG | Spino-cerebellar | SCA7 | K. sermon, AZ-VUB |
| | ataxia | | Bruxel BELGIUM |
| DMPK-CTG | Steinert's disease | VUB03 | K. sermon, AZ-VUB |
| | (DM1) | VUB19 | Bruxel BELGIUM |
| | | VUB24 | |

In one embodiment, the neural precursors are human neural precursors.

In one embodiment, the human neural precursors are obtained by a method which does not involve the destruction of a human embryo.

In one embodiment, the neural precursors are obtained according to the method described in WO2010/063848, i.e. they are obtained by culturing PSC in the presence of an inhibitor of the BMP signalling pathway, such as Noggin, and of an inhibitor of the TGF/activin/Nodal signalling pathway, such as SB431542.

Thus, in one aspect, the invention relates to method for obtaining neural precursors comprising the steps of:
culturing pluripotent cells in the presence of feeder cells;
preparing clusters of said pluripotent cells
culturing said clusters in the absence of feeder cells for several hours, in order to starve said pluripotent cells from the influence of the feeder cells;
plating the suspension in dishes coated with poly-ornithin and laminin in the presence of a Rock inhibitor;
replacing the medium every other day for with the medium comprising Noggin and SB431542 until neural rosettes are obtained (for examples, during 10 days);
dissociating said neural rosettes into one or several clusters of cells, mechanically or using an gentle enzymatic technique such as accutase;
plating said cluster(s) of cells in dishes coated with poly-ornithin and laminin and culturing said cells in the presence of a PKA inhibitor.

According to the method of the invention, the neural precursors can be cultured either as an adherent culture or as a non-adherent culture.

In one embodiment, the neural precursors are cultured as an adherent culture.

As used herein, the expression “adherent culture” refers to cultures conditions wherein the cells are attached to a substrate.

The substrate is typically a surface in a culture vessel, such as a flask or a plate or dish. In some embodiments, the substrate can be coated in order to improve the adhesion of the cells. Typically, the substrate can be coated with laminin, poly-ornithine, poly-lysine, gelatin or mixtures thereof.

In one embodiment, the cells are cultures on poly-ornithin/laminin coated dishes.

In another aspect, the invention also relates to a population of neural precursors obtainable by a method as defined above.

Advantageously, the population of neural precursors according to the invention is homogenous, i.e. it is not necessary to perform any sorting or selection to isolate the neural precursors from other contaminating cells. Typically, the population of neural stem cells according to the invention has a purity of at least 95%, preferably 99%, even more preferably 100%.

In one embodiment, the population of neural precursors obtained after amplification in the presence of a PKA inhibitor is a clonal population, i.e. a homogenous population of cells which are all derived from a single neural precursor.

The population of neural precursors according to the present invention can be used in a variety of applications.

Typically, the amplified neural precursors according to the invention can be used for substitutive cell therapy trials for treating ischemia, such as those described in Polentes et al., 2012.

Another application is the study of the pathological mechanisms involved in a variety of genetic diseases affecting the central nervous system, such as Huntington-Gilford progeria syndrome (Nissan et al., 2012) or Myotonic Dystrophy type 1 (Denis et al., 2013)

Alternatively, the population of neural precursors can be differentiated into precursors cells which are engaged into a specific lineage, such as the ventral mesencephalic precusors whic give rise to dopaminergic neurons involved in Parkinson's disease (Kriks et al., 2011; Carri et al., 2013)

In yet another aspect, the invention relates to a method for obtaining a population of neurons wherein said method comprises the steps of:

a) obtaining a population of neural precursors

b) amplifying said population of neural precursors in the presence of a PKA inhibitor

c) differentiating said population of neural stem cells into neurons.

The step consisting of differentiating neural stem cells into neurons can be carried according to techniques known to the skilled person (see for example Sun et al., 2008). For example, neural precursors can be derived into neural stems cells by plating onto plates coated with poly-ornithin/laminin and culture in the presence of BDNF.

The invention also relates to a population of neurons obtainable by the method described above.

The term “neuron” as used herein refers to fully differentiated, post-mitotic cells of the neural lineage. Neurons express the following markers: beta-3 tubulin (TUJ1 antigen), Microtubule Associated Protein 2 (MAP2), HuC/D antigen.

Typically, the population of neurons according to the invention has a purity of at least 40%, preferably 50%, even more preferably 60%.

The present invention also provides a pharmaceutical composition comprising the population of neural precursors or population of neurons according to the invention. The pharmaceutical composition may generally include one or more pharmaceutically acceptable and/or approved carriers, additives, antibiotics, preservatives, adjuvants, diluents and/or stabilizers. Such auxiliary substances can be water, saline, glycerol, ethanol, wetting or emulsifying agents, pH buffering substances, or the like. Suitable carriers are typically large, slowly metabolized molecules such as proteins, polysaccharides, polylactic acids, polyglycollic acids, polymeric amino acids, amino acid copolymers, lipid aggregates, or the like. This pharmaceutical composition can contain additional additives such as mannitol, dextran, sugar, glycine, lactose or polyvinylpyrrolidone or other additives such as antioxidants or inert gas, stabilizers or recombinant proteins (e. g. human serum albumin) suitable for in vivo administration.

As used herein, the term “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

Another aspect of the invention relates to a population of neural precursors of the invention or a population of neurons as described above, for use in treating a neurodegenerative disease or a brain injury.

The invention also relates to a method for treating a neurodegenerative disease or brain injury comprising the step of administering a pharmaceutically effective amount of a population of neural precursors of the invention or a population of neurons as described above to a patient in need thereof.

In the context of the invention, the term “treating” or “treatment”, as used herein, refers to a method that is aimed at delaying or preventing the onset of a pathology, at reversing, alleviating, inhibiting, slowing down or stopping the progression, aggravation or deterioration of the symptoms of the pathology, at bringing about ameliorations of the symptoms of the pathology, and/or at curing the pathology.

As used herein, the term “pharmaceutically effective amount” refers to any amount of neural precursors or neurons according to the invention (or a population thereof or a pharmaceutical composition thereof) that is sufficient to achieve the intended purpose.

Effective dosages and administration regimens can be readily determined by good medical practice based on the nature of the pathology of the subject, and will depend on a number of factors including, but not limited to, the extent of the symptoms of the pathology and extent of damage or degeneration of the tissue or organ of interest, and characteristics of the subject (e.g., age, body weight, gender, general health, and the like).

For therapy, neural precursors, neurons and pharmaceutical compositions according to the invention may be administered through intracerebral route. The dose and the number of administrations can be optimized by those skilled in the art in a known manner.

In one embodiment, the neurodegenerative disease or brain injury is selected from the group consisting of retinopathy, Huntington's disease, Spino-cerebellar ataxia, Steinert's disease, Parkinson's disease, Alzheimer's disease and cerebral ischemia, Multiple sclerosis, Amyotrophic lateral sclerosis, Traumatic Brain Injuries and Myotonic Dystrophy.

Yet another aspect of the invention relates to a method for screening compounds having a neuroprotective and/or neurotoxic effect wherein said method comprises the steps of:
a) culturing a population of neural precursors or a population of neurons of the invention in the presence of a test compound;
b) comparing the survival of the cells cultured in step a) to that of a population of neural precursors or a population of neurons as defined above cultured in the absence of said test compound.

The term “neurotoxic” refers to a compound which provokes a decrease in the survival of neural precursors or neurons. A compound is deemed to have a neurotoxic effect if the number of viable cells cultured in the presence of said compound is lower than the number of viable cells cultured in the absence of said compound.

The term “neuroprotective” refers to a compound which results in an increase survival of neural precursors or neurons. A compound is deemed to have a neuroprotective effect if the number of viable cells cultured in the presence of said compound is higher than the number of viable cells cultured in the absence of said compound. Typically, the neuroprotective effect can be assayed in the absence of neurotrophic factors. Alternatively, the neuroprotective effect can be assayed in the presence of a known neurotoxic drug.

The invention will be further illustrated through the following example and figures.

### FIGURES

FIG. 1: Typical results obtained after screening 80 kinase inhibitors for their ability to sustain neural precursor proliferation. Compound-treated cells were compared to DMSO-treated cells. Percentage of Ki67 positive cells was representative of the percentage of proliferating precursors after 7 days without growth factors.

FIG. 2: Dose-response experiments with three PKA inhibitors.

FIG. 3: Reduction by a PKA activator of neural precursor proliferation

FIG. 4: Comparative amplification curves of cells cultivated using the reference EFB medium or different PKA inhibitors.

FIG. 5: Differentiation potential of cells amplified in the EFB reference medium or in presence of the PKA inhibitor HA1004. HuCD positive cells represent post-mitotic neurons whereas Ki67/SOX2 positive cells represent cycling undifferentiated neural precursors.

FIG. 6: Neural precursors derived directly in PKAi medium present the same morphology as precursors derived in the reference medium EFB and express the neural marker nestin.

### EXAMPLE

### Material and Methods

### Media and Cytokines

N2B27 medium was described in Ying et al., 2003. N2B27 was a mixture of DMEM-F12/Neurobasal 1:1, N2 supplement)(1:100°, B27 supplement)(1:50° both obtained from Life Technologies. NFS was composed of N2B27, Noggin (range of concentration between 2 500 ng/ml, from Preprotech), SB431542 (between 20 μM, from Tocris), 5 ng/ml FGF2 (Preprotech). Rock inhibitor Y27632 was from Stemgene, EGF and BDNF were from RD systems.

### Human Pluripotent Stem Cells

Different pluripotent stem cell lines were used in this study. Wild type SA001 cells (karyotype 46, XY) were from Cellartis. WO9 cells (karyotype 46, XX) were from Wicell. Induced pluripotent stem cell (iPS cell) lines were also used.

### Human Pluripotent Stem Cell (PSC) Culture.

Human PSC were maintained on a layer of inactivated mouse fibroblasts. The human PSC were cultured in DMEM/F12/Glutamax supplemented with 20% knockout serum replacement (KSR), 1 mM nonessential amino acids, 0.55 mM 2-mercaptoethanol, and 10 ng/ml recombinant human FGF2 (all from Invitrogen). Cultures were fed daily and manually passaged every 5-7 days. The cells were used between passages 40 and 60.

### Neural Induction Using NFS Medium

Human PSC cultures reaching 70-80% confluence were used to perform neural induction using NFS medium. Colonies were cut in pieces and manually detached in NFS medium completed with the Rock-inhibitor Y27632 (10 μM, Calbiochem). Clusters were transferred for 6 h in a low attachment Petri dish in order to completely starve them from the influence of the feeders. Finally, the PSC suspension was plated in the same medium at a 1:1 ratio in culture dishes pre-coated with poly-ornithin and laminin (PO/lam mixture; laminin at 2 μg/ml, Life technologies). The day after and then every other day, the medium was changed for NFS medium without Y27632.

### Amplification of Neural Precursors

After two weeks of differentiation, neural rosettes containing SOX1 positive cells were cut mechanically or using a gentle enzymatic technique (accutase or equivalent), in order to transfer small clusters of cells (about 100 cells) into new tissue culture dishes coated with PO/lam. Amplification was performed using PKAi medium (N2B27+PKA inhibitor such as HA 1004 at 40 μM+Laminin at 2 μg/ml). From passage 2, cells were detached as a unicellular suspension using trypsin and seeded at 100 000 cell/cm².

As a reference for neural precursor amplification, neural precursors were grown in EFB medium (N2B27+FGF-2 (10 ng/ml)/EGF (10 ng/ml)/BDNF (20 ng/ml), as described in WO2010/063848.

This medium was described previously (Conti et al., 2005) and its amplification properties were based on the use of a combination of two mitogens namely EGF (Epidermal Growth factor) and FGF2 (Fibroblast Growth factor 2) and the optional addition of the survival factor BDNF (Brain-derived Growth Factor).

This medium was used in the authors' previous study

Terminal Differentiation into Neurons

Terminal differentiation into neurons was induced by plating neural precursors on poly-ornithin/laminin at a density of 50,000 cells/cm² in N2B27 medium. Medium was changed every 4 days.

### FACS Analysis

Cells were collected using trypsin and fixed with 2% PFA for 15 min at 4° C. Permeabilization was performed using a PBS/0.1% saponin solution 10 min at RT. The same solution was then used to dilute primary antibodies. Cells were exposed to the mixture of primary antibodies 2 h at RT. An additional incubation with secondary antibodies linked to AlexaFluo 488 was performed during 1 h at RT. FACS analysis was performed using a Milteny flow cytometer.

### Immuno-Cytochemistry

Cells were fixed with 4% PFA for 15 min at 4° C. then permeabilized using a PBS/0.3% Triton X100 solution, 10 min at RT. Incubation with primary antibodies was performed. AlexaFluor secondary antibodies and DAPI counterstaining were applied for 1 h at room temperature. Detection was performed using a Zeiss Inverted microscope or the Arrayscan automated microscope.

Real-Time qPCR.

Total RNA was isolated using the RNeasy Mini Kit (Qiagen) according to the manufacturer's instructions. A total of 500 ng of RNA were reverse transcribed into cDNA with SuperScript III (Invitrogen) using random primers. Real-time Q-PCR was performed with SYBR Green as a probe on a LC480 Real-Time system (Roche). Quantification was performed at a threshold detection line (Cₜ value). The Cₜ of each target gene was normalized against that of the RNA18s as a housekeeping gene. The 2^{-ΔΔCt} method was used to determine the relative level of expression of each gene. Data were expressed as mean±SEM.

### Results

A homogeneous population of neural precursors was produced after conversion of SA001 PSC line into neural cells and further amplification using EFB medium until passage 8. The stable neural precursors were seeded in 384 well-plates using Agilent BRAVO automate in N2B27 without growth factors. Six hours post-seeding, cells were treated with Enzo Life Sciences Kinase inhibitors library (BML-2832), a collection of 80 different kinase inhibitors. Another treatment was performed at day 4 and the percentage of proliferative precursors was quantified at day 7 using Ki67 and EdU incorporation. SOX2 was used as a neural marker.

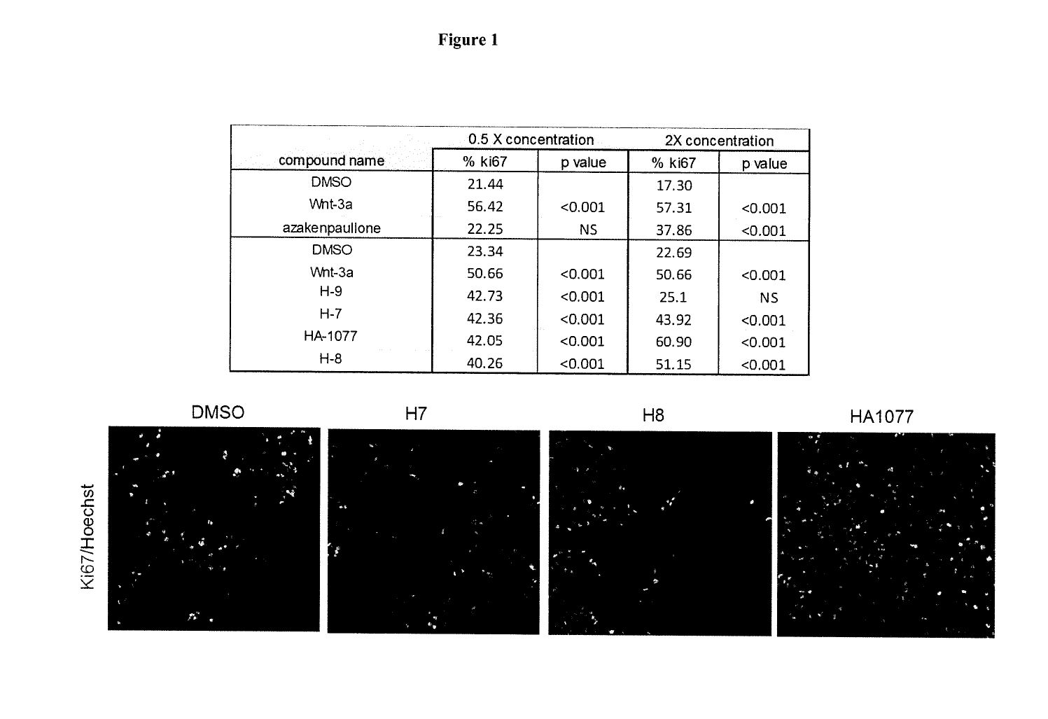

This screening revealed that several molecules described as PKA inhibitors (H9, HA1077, H7, H8) sustained neural precursor self-renewal (FIG. 1).

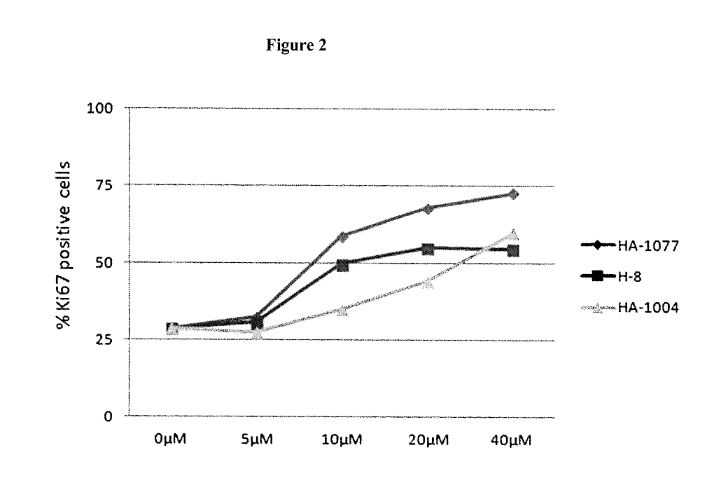

To challenge the robustness of the results obtained during this primary screening, several compound described as PKA inhibitors were re-tested at several doses. These experiments confirmed that PKA inhibitors dose-dependently regulated neural precursor self-renewal and proliferation (FIG. 2).

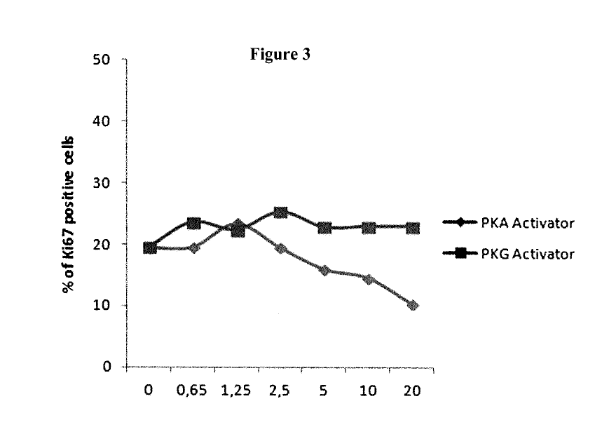

To validate that each molecule acted by inhibiting PKA, a mirror experiment was conducted treating the cells with an activator of PKA (FIG. 3). This treatment led to a significant inhibition of proliferation of neural precursors, indicating that PKA is indeed a pivotal enzyme in the control of neural precursor cells self-renewal.

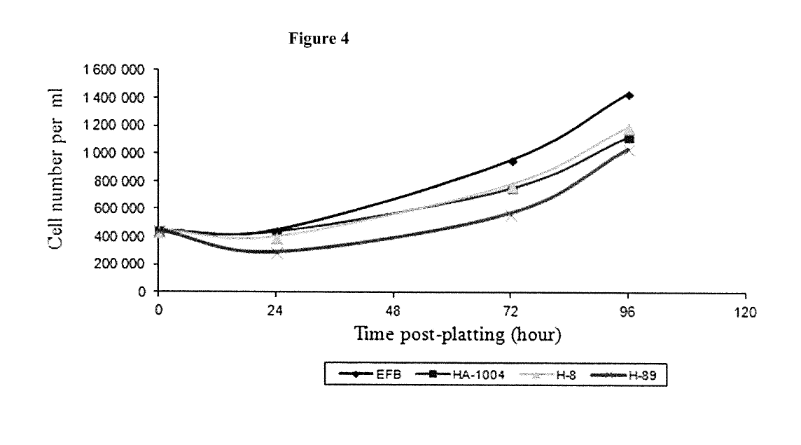

Amplification rates of neural precursors amplified using different PKA was compared to amplification rates of neural precursor amplified in the reference medium EFB (FIG. 4). This demonstrated that a robust amplification is obtained using PKA inhibitors. We decided to further investigate their potential using the molecule HA1004.

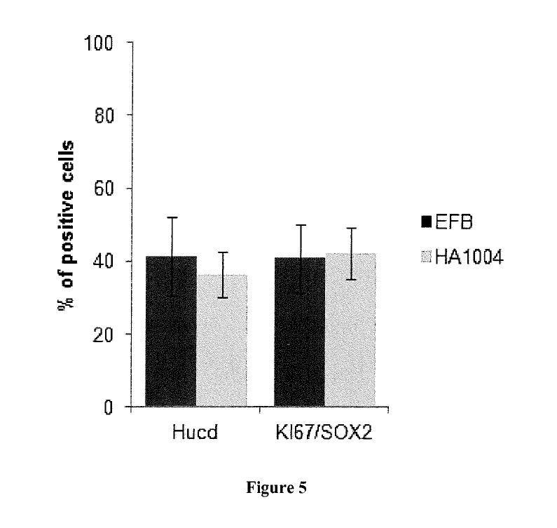

We next measured the ability of neural precursors amplified using the PKA inhibitor HA1004 for 5 passages to produce neurons. This was compared to the production of neurons obtained from precursor amplified using EFB medium as a reference. These experiments demonstrated that long term amplification of neural precursors in PKAi medium did not impact on their ability to produce neurons (FIG. 5).

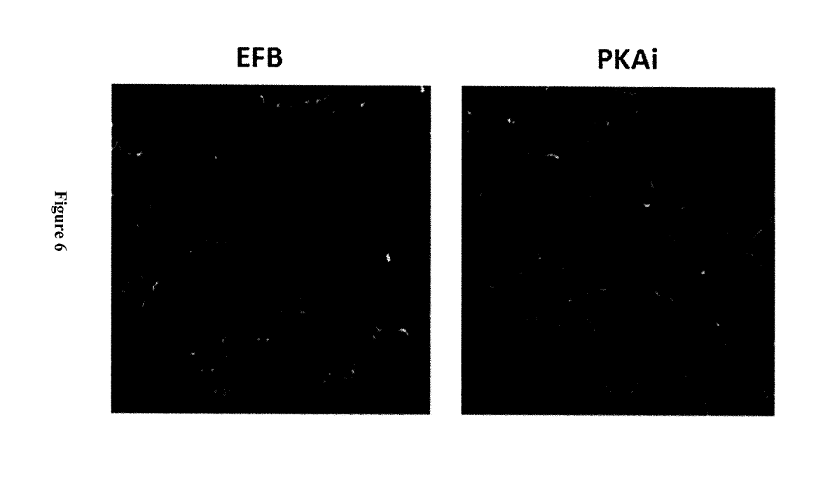

We finally asked whether a stable and homogeneous population of neural precursors could be derived from PSC by directly by-passing the use of EFB medium. Neural rosettes containing SOX1-positive precursors were obtained after 10 days of differentiation of PSC, then directly seeded and amplified in PKAi medium instead of EFB medium. After 5 passages, the resulting neural precursors exhibited the same morphology as cells derived using EFB, expressed typical neural markers (like Nestin) and produced neurons upon differentiation (FIG. 6).

Taken together, these experiments demonstrated that PKA inhibitors can fully replace complex growth factor cocktail to amplify PSC-derived neural precursors.

Similar results were obtained using the WO9 PSC line or induced pluripotent stem cells (iPS) as starting material.

### REFERENCES

Throughout this application, various references describe the state of the art to which this invention pertains. The disclosures of these references are hereby incorporated by reference into the present disclosure.
Carri A D, Onorati M, Lelos M J, Castiglioni V, Faedo A, Menon R, Camnasio S, Vuono R, Spaiardi P, Talpo F, Toselli M, Martino G, Barker R A, Dunnett S B, Biella G, Cattaneo E. Developmentally coordinated extrinsic signals drive human pluripotent stem cell differentiation toward authentic DARPP-32+ medium-sized spiny neurons. Development. 2013 Jan. 15; 140(2):301-12
Chen B Y, Wang X, Wang Z Y, Wang Y Z, Chen L W, Luo Z J. Brain-derived neurotrophic factor stimulates proliferation and differentiation of neural stem cells, possibly by triggering the Wnt/β-catenin signaling pathway. J Neurosci Res. 2013 January; 91(1):30-41.
Cheng H C, Kemp B E, Pearson R B, Smith A J, Misconi L, Van Patten S M, Walsh D A. A potent synthetic peptide inhibitor of the cAMP-dependent protein kinase. J Biol Chem. 1986 Jan. 25; 261(3):989-992
Conti L, Pollard S M, Gorba T, Reitano E, Toselli M, Biella G, Sun Y, Sanzone S, Ying Q L, Cattaneo E, Smith A. Niche-independent symmetrical self-renewal of a mammalian tissue stem cell. PLoS Biol. 2005 September; 3(9):e283.
Denis J A, Gauthier M, Rachdi L, Aubert S, Giraud-Triboult K, Poydenot P, Benchoua A, Champon B, Maury Y, Baldeschi C, Scharfmann R, Piétu G, Peschanski M, Martinat C. mTOR-dependent proliferation defect in human ES-derived neural stem cells affected by Myotonic Dystrophy Typel. J Cell Sci. 2013 Feb. 26. [Epub ahead of print]
Dumaz N, Marais R: Protein kinase A bloks Raf-1 activity by stimulating 14-3-3 binding and blocking Raf-1 interaction with Ras. J Biol Chem 2003; 278: 29819-23.
Freemerman A J, Turner A J, Birrer M J, Szabo E, Valerie K, Grant S: Role of c-jun in human myeloid leukaemia cell apoptosis induced by pharmacological inhibitors of protein kinase C. Mol Pharmacol 1996; 49: 788-95.
Gjertsen B T, Mellgren G, Otten A, Maronde E, Genieser H G, Jastorff B, Vintermyr O K, McKnight G S, Doskeland S O: Novel (Rp)-cAMPS analogs as tools for inhibition of camp-kinase in cell culture. J Biol Chem 1995; 270:20599-607.
Glass D B, Lundquist L J, Katz B M, Walsh D A: Protein kinase inhibitor-(6-22)-amide peptide analogs with standard and non-standard amino acid substitutions for phenylalanine 10. J Biol Chem 1989; 264: 14579-84.
Hidaka H, Inagaki M, Kawamoto S, Saaki Y: Isoquinolinesulfonamides, novel and potent inhibitors of cyclic nucleotide dependent protein kinase and protein kinase C. Biochemistry 1984; 23: 5036-41.
Kriks S, Shim J W, Piao J, Ganat Y M, Wakeman D R, Xie Z, Carrillo-Reid L, Auyeung G, Antonacci C, Buch A, Yang L, Beal M F, Surmeier D J, Kordower J H, Tabar V, Studer L. Dopamine neurons derived from human ES cells efficiently engraft in animal models of Parkinson's disease. Nature. 2011 Nov. 6; 480(7378):547-51
Liu Y, Song Z, Zhao Y, Qin H, Cai J, Zhang H, Yu T, Jiang S, Wang G, Ding M, Deng H. A novel chemical-defined medium with bFGF and N2B27 supplements supports undifferentiated growth in human embryonic stem cells. Biochem Biophys Res Commun. 2006 Jul. 21; 346(1):131-9.
Lowell S, Benchoua A, Heavey B, Smith A G. Notch promotes neural lineage entry by pluripotent embryonic stem cells. PLoS Biol. 2006 May; 4(5)
Monaghan T K, MacKenzie C J, Plevin R, Ltz E M: PACAP-38 induces neuronal differentiation of human SH-SY5Y neuroblastoma cells via camp-mediated activation of ERK and p38 MAP kinases. J Neurochem 2008; 104: 74-88.
Murray A J: Pharmacological PKA inhibition: all may not be what it seems. Sci Signal 2008; 1(22):re4.
Nissan X, Blondel S, Navarro C, Maury Y, Denis C, Girard M, Martinat C, De Sandre-Giovannoli A, Levy N, Peschanski M. Unique preservation of neural cells in Hutchinson-Gilford progeria syndrome is due to the expression of the neural-specific miR-9 microRNA. Cell Rep. 2012 Jul. 26; 2(1):1-9. doi: 10.1016/j.celrep.2012.05.015. Epub 2012 Jun. 21.
Ogreid D, Dostmann W, Genieser H G, Niemann P, Doskeland S O, Jastorff B: (Rp)- and (Sp)-8-piperidino-adenosine 3′,5′-(cyclic)thiophosphates discriminate completely between site A and B of the regulatory subunits of camp-dependent protein kinase type I and II. Eur J Biochem 1994; 221: 1089-94.
Qiu L B, Ding G R, Li K C, Wang X W, Zhou Y, Zhou Y C, Li Y R, Guo G Z: The role of protein kinase C in the opening of blood-brain barrier induced by electromagnetic pulse. Toxicology 2010; 273: 29-34.
Rudolph J A, Pratt J, Mourya R, Steinbrecher K A, Cohen M B: Novel mechanism of cyclic AMP mediated extracellular signal regulated kinase activation in an intestinal cell line. Cell Signal 2007; 19: 1221-8.
Ruiz-Velasco V, Zhong J, Hume J R, Keef K D: Modulation of Ca2+ channels by cyclic nucleotide cross activation of opposing protein kinases in rabbit portal vein. Circ Res 1998; 82: 557-65.
Sun Y, Pollard S, Conti L, Toselli M, Biella G, Parkin G, Willatt L, Falk A, Cattaneo E, Smith A. Long-term tripotent differentiation capacity of human neural stem (NS) cells in adherent culture. Mol Cell Neurosci. 2008 June; 38(2):245-58
Tabar V, Panagiotakos G, Greenberg E D, Chan B K, Sadelain M, Gutin P H, Studer L. Migration and differentiation of neural precursors derived from human embryonic stem cells in the rat brain. Nat Biotechnol. 2005 May; 23(5):601-6.
Takahashi K. and Yamanaka S. Induction of pluripotent stem cells from mouse embryonic and adult fibroblast cultures by defined factors. Cell. 2006 Aug. 25; 126(4):663-76
Takahashi K, Tanabe K, Ohnuki M, Narita M, Ichisaka T, Tomoda K, Yamanaka S. Cell. 2007 Nov. 30; 131(5):861-72.
Wang L Y, Salter M W, MacDonald J F: Regulation of kainite receptors by cAMPD-dependent protein kinase and phosphatases. Science 1991; 253: 1132-35.
Weisskopf M G, Castillo P E, Zalutsky R A, Nicoll R A: Mediation of hippocampal mossy fiber long-term potentiation by cyclic AMP. Science 1994; 265: 1878-82.
Ying Q L, Stavridis M, Griffiths D, Li M, Smith A. Conversion of embryonic stem cells into neuroectodermal precursors in adherent monoculture. Nat Biotechnol. 2003 February; 21(2):183-6.
Yokozaki H, Tortora G, Pepe S, Maronde E, Genieser H G, Jastorff B, Cho-Chung Y S: Unhydrolyzable alalogues of adenosine 3′:5′-monophosphate demonstrating growth inhibition and differentiation in human cancer cells. Cancer Res 1992; 52: 2504-8.
Yu J, Vodyanik M A, Smuga-Otto K, Antosiewicz-Bourget J, Frane J L, Tian S, Nie J, Jonsdottir G A, Ruotti V, Stewart R, Slukvin I I, Thomson J A.
Zhang B, Zhang Y, Shacter E: Rac1 inhibits apoptosis in human lymphoma cells by stimulating Bad phosphorylation on Ser-75. Mol C. 2004
Zhang Q, Liu G, Wu Y, Sha H, Zhang P, Jia J. BDNF promotes EGF-induced proliferation and migration of human fetal neural stem/progenitor cells via the PI3K/Akt pathway. Molecules. 2011 Dec. 6; 16(12):10146-56

1. Method for amplifying a population of neural precursors comprising the step of culturing neural precursors in the presence of a PKA inhibitor.

2. The method according to claim 1 wherein said PKA inhibitor is a selective PKA inhibitor.

3. The method according to claim 1 wherein said PKA inhibitor is a non-selective PKA inhibitor.

4. The method according to claim 1 wherein said PKA inhibitor is selected from the group consisting of H7, H8, H9, H89, HA 1077 and HA 1004.

5. The method according to claim 1 wherein said PKA inhibitor is HA 1004 and is used at a concentration comprised between 20 μM and 40 μM.

6. The method according to claim 1, wherein the step of culturing neural precursors is carried out in a culture medium comprising:
a source of carbon as energy substrate, such as glucose, galactose or sodium pyruvate;
essential amino-acids;
at least one vitamin;
at least a purine and a pyrimidine as nucleic acid precursors;
inorganic salts;
at least one molecule known to limit natural ageing;
at least one antioxidant and/or at least one enzyme involved in Reactive Oxygen Species detoxification;
at least one phospholipid precursor;
at least one unique fatty acid;
at least one carrier protein; and
optionally, at least one additional protein or peptide.

7. The method according to claim 1, wherein said culture medium is N2B27 medium comprising DMEM/F12 and Neurobasal media in a 1/1 ratio, N2 supplement (1/100), B27 supplement (1/50) and beta-mercaptoethanol (1/1000).

8. The method according to claim 1, wherein the neural precursors are human neural precursors.

9. The method according to claim 1, wherein the neural precursors are derived from embryonic stem cells (ES cells) or from induced pluripotent stem cells (iPS cells).

10. A method for obtaining neural precursors comprising the steps of:
culturing pluripotent cells in the presence of feeder cells;
preparing clusters of said pluripotent cells
culturing said clusters in the absence of feeder cells for several hours, in order to starve said pluripotent cells from the influence of the feeder cells;
plating a suspension of said clusters in dishes coated with poly-ornithin and laminin in the presence of a Rock inhibitor;
replacing the medium every other day for with the medium comprising Noggin and SB431542 until neural rosettes are obtained;
dissociating said neural rosettes into one or several clusters of cells, mechanically or using an gentle enzymatic technique;
plating said cluster(s) of cells in dishes coated with poly-ornithin and laminin and culturing said cells in the presence of a PKA inhibitor.

11. (canceled)

12. Kit for the culture of neural precursors, comprising a culture medium and a PKA inhibitor.

13. A population of neural precursors obtained by a method as defined in claim 1.

14. A method for treating a neurodegenerative disease or a brain injury in a subject in need thereof comprising the steps of
administering to said subject a population of neural precursors obtained by the method of claim 1.

15. A method for screening compounds having a neuroprotective and/or neurotoxic effect wherein said method comprises the steps of:
a) culturing a population of neural precursors obtained by the method of claim 1 in the presence of a test compound; and
b) comparing the survival of the cells cultured in step a) to that of a population of neural precursors or a population of neurons as defined above cultured in the absence of said test compound.

16. The method of claim 6, wherein
i) said at least one vitamin is selected from the group consisting of biotin, folic acid, and B12;
ii) said at least one molecule known to limit natural ageing is selenium;
iii) said at least one antioxidant is glutathione reduced (GSH) or ascorbic acid and said enzyme involved in Reactive Oxygen Species detoxification is catalase or superoxide dismutase;
iv) said at least one phospholipid precursor is selected from the group consisting of choline, inositol;
v) said at least one unique fatty acid is selected from the group consisting of linoleic acid, linolenic acid and a lipoic acid;
vi) said at least one carrier protein is albumin or heparin; and,
vii) said at least one additional protein or peptide is selected form the group consisting of insulin, transferrin and an agonist of the IGF-1 receptor.

17. The method of claim 16, wherein said cholesterol derivative is corticosterone.

18. The method of claim 10, wherein said gentle enzymatic technique is an accutase technique.
